(19) United States
(12) Patent Application Publication
(10) Pub. No.: US 2024/0366287 A1
(43) Pub. Date: Nov. 7, 2024

(54) LAPAROSCOPIC SUCTION, IRRIGATION, AND GRASPER TOOL

(71) Applicant: KING FAISAL UNIVERSITY, Al-Ahsa (SA)

(72) Inventor: Abdulrahman Saleh ALMULHIM, Al-Ahsa (SA)

(21) Appl. No.: 18/143,152

(22) Filed: May 4, 2023

(51) Int. Cl. A61B 18/14

(52) U.S. Cl. CPC A61B 18/1445; A61B 2017/2925; A61B 2017/2929; A61B 2018/00589; A61B 2218/002; A61B 2218/007

(57) ABSTRACT

A multifunctional laparoscopic suction and irrigation surgical grasping tool using a laparoscope, an endoscope, colonoscope, or gastroscope for the deployment of a grasper with diathermy, suction, and irrigation into a surgical field of view. Once optimally positioned, the grasper can remove tissue while providing a coagulating electric charge to the area from which the tissue is removed. Suction and irrigation can also be selectively deployed as needed during the tissue removal process.

### BACKGROUND

### 1. Field

The present disclosure relates a surgical tool used in laparoscopic medical procedures and particularly, those which require suction, irrigation, and grasping.

### 2. Description of the Related Art

Laparoscopic surgery is a favored surgical method because it remains minimally invasive as a procedure. In particular, it is seen as a desired alternative to open surgical procedures and as such, this intracavity surgical method has replaced a number of past open surgery procedures. The traditional method of laparoscopic surgery is to make 3-5 small incisions of about 1 cm each in the abdomen of a patient, insert a pipeline-shaped working channel called a Trocar, and continuously inject CO₂ with constant pressure into the abdominal cavity. The gas creates a visual space allowing for all of the later operations to be conducted through the pipelines, and as a result, the same steps for an open surgery procedure can be completed by a specially lengthened surgical instrument comprising a camera and associated surgical tools.

However, most laparoscopic instruments have limited functionality and often times only allow for one basic tool to be deployed at the end of a laparoscope. However, during an intracavity surgery procedure, multiple instruments may be needed to conduct the functions of suction for when excessive bleeding needs to be sucked up. There may be a need to have a grasping tool on the end of the laparoscope for performing a grasping function within the surgical field. Other tool based functions include irrigation, dissection, and diathermy. Furthermore, a single operation often needs multiple tools to be installed and replaced throughout the procedure which tends to unnecessarily prolong the operation time. Additionally, if the surgical field of view is unnecessarily polluted and obscured by unwanted bleeding, the operation efficacy is deleteriously affected, and further the operational safety is negatively impacted.

What is needed is a multifunctional laparoscopic instrument that has a plurality of tools available to the medical practitioner so that the most frequent functions conducted during an intracavity surgery operation are readily available to the medical practitioner so that the laparoscope can remain operative and within the surgical field of view while switching from one surgical function to another, thus reducing operation time.

### SUMMARY

In a first aspect and in order to achieve the above object, the present subject matter provides the following multifunctional tools, all in one integrated device: tissue grasping forceps with diathermy, suction, and irrigation. The multifunctional laparoscopic suction and irrigation surgical grasping tool comprises a fixed handle, wherein the fixed handle has a hollow structure, and wherein a topside of the fixed handle is provided with three vertical post like protrusions comprising or consisting of a suction button, an irrigation button, and a diathermy connector. A grasper handle extends from the fixed handle's hollow structure downwardly and at an angle from the fixed handle grip portion. Furthermore, for the grasper handle, a connecting hub is arranged in the fixed handle such that the connecting hub is rotatably connected to an end of the grasper handle. One side of the fixed handle terminates in a rotating grasper collar which is connected to the fixed handle through a bearing (not shown). The rotating grasper collar has one side connected to a long, hollow pipe like structure having a distal end and a proximal end and containing therein suction and irrigation (i.e., for flushing) respective pipelines, and overlaying the suction and irrigation pipelines is a channel housing a grasper with diathermy. The grasper is activated by squeezing the grasper handle to perform the grasping tool's function. The distal end of the laparoscope includes a hole for the grasping forceps and respective holes for the suction and irrigation pipes.

In another embodiment, the present subject matter relates to a method of assisting in laparoscopic surgery in a patient, the method comprising: making an incision in a surgical site in a body of the patient; inserting a device having a grasper through the incision into the body of the patient, the device comprising: a fixed handle including a suction button, an irrigation button, and a diathermic connector on a topside of the fixed handle; a hollow long pipe including an interior, a distal end and a proximal end; a rotating grasper collar disposed on an end of the fixed handle connecting the fixed handle to the proximal end of the long pipe, wherein the rotating grasper collar is connected to the fixed handle with a bearing; a grasper handle; and a grasper extending from the distal end of the long pipe, wherein a rotation of the rotating grasper collar with respect to an axis of the long pipe causes the grasper to change an angular orientation of the grasper; squeezing the grasper handle to move the grasper handle closer to the fixed handle, thereby producing a firing action, wherein the firing action both activates an electric charge through the diathermic connector that is carried along the grasper and closes jaws of the forceps shut around tissue for removal in the body of the patient at the surgical site; and retracting the grasper back towards the distal end of the long pipe, thereby removing the tissue for removal from the body of the patient at the surgical site; wherein the electric charge carried along the grasper induces electric coagulation within tissue to remain in the body of the patient adjacent to the tissue for removal at the surgical site, thereby reducing tissue bleeding in the tissue to remain in the patient at the surgical site.

These and other features of the present subject matter will become readily apparent upon further review of the following specification.

### BRIEF DESCRIPTION OF THE DRAWINGS

To clearly illustrate the embodiments of the present subject matter, the drawings used in this description will be briefly described below. It should be apparent that the drawings in the following description are merely exemplary, and that other embodiments can be derived from the drawings provided by those of ordinary skill in the art. The structure, ratio, size, and the like shown in the present specification are used only for matching with the content disclosed in the specification, to be known and read by people familiar with the technology and are not used for limiting the conditions which with the present subject matter can be practiced. Any structural modification, ratio relationship change, or size adjustment should still fall within the scope of the present subject matter and can be covered by the technical content disclosed herein without affecting the efficacy and achievable purpose of the present subject matter.

FIG. 1 is a diagram of a side view of the multifunctional laparoscopic grasping tool.

FIG. 2 is a diagram of a side view of the multifunctional laparoscopic grasping tool with additional connection details within the fixed handle.

Similar reference characters denote corresponding features consistently throughout the attached drawings.

### DETAILED DESCRIPTION OF EMBODIMENTS

The following detailed description is merely exemplary in nature and is not intended to limit the described embodiments or the application and uses of the described embodiments. Any implementation described herein with the words “exemplary” or “illustrative” is not necessarily construed as preferred or advantageous over other implementations. All of the implementations described below are exemplary implementations provided to enable persons skilled in the art to make or use the embodiments of the disclosure and are not intended to limit the scope of the disclosure, which is defined by the claims. For the purposes of the description herein, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the concepts defined in the appended claims. Hence, specific dimensions and other physical characteristics relating to the embodiments disclosed therein are not to be considered as limiting, unless the claims expressly state otherwise.

In order to solve the technical problem that exists as discussed above, this disclosure provides a multifunctional laparoscopic suction and irrigation surgical grasping tool that provides the advantage of streamlining an intracavity surgical procedure using a laparoscope, aims at a drastic reduction in surgical tool changes to the apparatus, provides convenience to the medical practitioner in performing the procedure, and reduces the risk of operational infections by reducing the surgical procedure time.

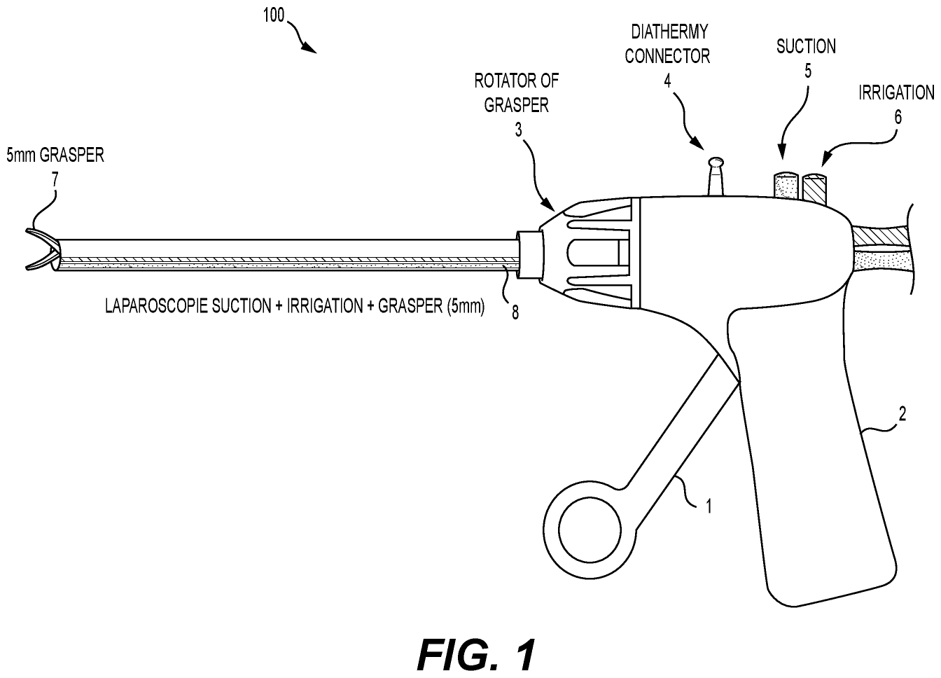

Referring to FIG. 1, the multifunctional laparoscopic suction and irrigation surgical grasping tool (100) comprises a fixed handle (2), wherein the fixed handle has a hollow structure, and wherein the handle is generally in the shape of an inverted L. A rotating grasper collar (3) is disposed on one side of the fixed handle (2) and is connected with the fixed handle through a bearing (not shown). A long pipe is arranged on the other side of the rotating grasper collar (3) such that the long pipe extends distally away from the fixed handle. The rotating grasper collar (3) when rotated about the axis of the long pipe allows for a change in the angular orientation of a grasper (7), which in this instance are a pair of grasping forceps (7) extending outward from the distal end of the pipe of the multifunctional laparoscopic suction and irrigation surgical grasping tool (100) in a V shape such that the distance between the jaws of the grasping forceps (7) is 5 mm.

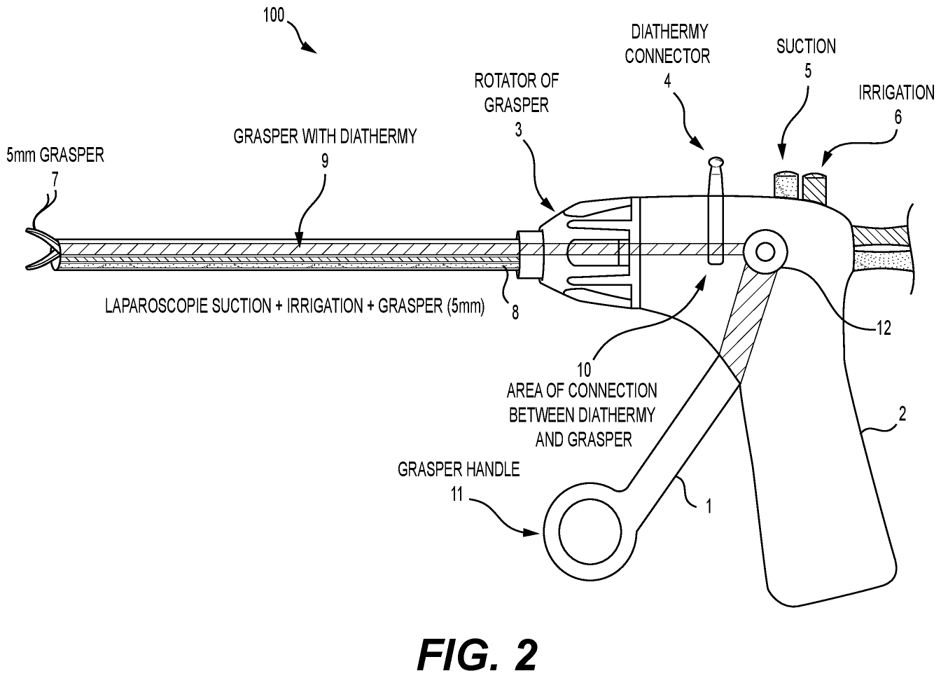

As shown in FIG. 2, a more detailed view of the interior of the long pipe extending distally from the rotating grasper collar (3) of the fixed handle (2), and further detail into the hollow structure of the fixed handle (3) are provided. The interior of the long pipe includes a suction and irrigation pipe pair (8) with intake and discharge holes (not shown) respectively, at the distal end of the long pipe for providing suction and irrigation/flushing at the surgical site. Atop the suction and irrigation pipe pair (8) is a channel housing the grasping forceps (7). The grasping forceps are hinged above the intake and discharge holes for suction and irrigation/flushing at the surgical site. Additionally, the grasping forceps (7) are comprised of an electrically conductive material, so that an electric charge for diathermy purposes can be provided through the grasping forceps to the tissue being grasped, in use, to induce electric coagulation within the relevant tissue, and thus reduce tissue bleeding. This provision of an electrical charge can be realized by attaching at the diathermy connector (4) any suitable electrosurgical equipment sufficient to provide the appropriate electrical charge.

As shown in FIG. 2, the diathermy connector (4) contacts the grasper (5) at a point of intersection (10) between the two elements. Also shown is a connection between the grasper handle (11) and the fixed handle (2) at a connection hub (12). When a squeezing motion is provided by a medical practitioner's hand (not shown) causing the stem (1) of the grasper handle (11) to be moved closer to the fixed handle (2), a firing action is provided to the grasping forceps (7). This firing action causes the grasping forceps (7) to close and retract such that the removal of tissue within the closed jaws of the forceps does not lead to exacerbated bleeding based on the fact that a diathermic charge was also applied while initiating the firing action with the squeezing of the grasping handle (11). After the tissue has been removed, suction can be provided by depressing the suction button (5) such that the any excess blood can be removed. Likewise, the relevant area can be washed out with irrigation provided by pushing down the irrigation button (4), thereby cleaning out wound area and ensuring that infection risks are minimized.

In another embodiment, the present subject matter relates to a method of assisting in laparoscopic surgery in a patient, the method comprising: making an incision in a surgical site in a body of the patient; inserting a device having a grasper through the incision into the body of the patient, the device comprising: a fixed handle including a suction button, an irrigation button, and a diathermic connector on a topside of the fixed handle; a hollow long pipe including an interior, a distal end and a proximal end; a rotating grasper collar disposed on an end of the fixed handle connecting the fixed handle to the proximal end of the long pipe, wherein the rotating grasper collar is connected to the fixed handle with a bearing; a grasper handle; and a grasper extending from the distal end of the long pipe, wherein a rotation of the rotating grasper collar with respect to an axis of the long pipe causes the grasper to change an angular orientation of the grasper; squeezing the grasper handle to move the grasper handle closer to the fixed handle, thereby producing a firing action, wherein the firing action both activates an electric charge through the diathermic connector that is carried along the grasper and closes jaws of the forceps shut around tissue for removal in the body of the patient at the surgical site; and retracting the grasper back towards the distal end of the long pipe, thereby removing the tissue for removal from the body of the patient at the surgical site; wherein the electric charge carried along the grasper induces electric coagulation within tissue to remain in the body of the patient adjacent to the tissue for removal at the surgical site, thereby reducing tissue bleeding in the tissue to remain in the patient at the surgical site.

In certain embodiments of the present methods, the device further comprises a suction pipe and an irrigation pipe within the long pipe, such that the grasper, the suction pipe, and the irrigation pipe are all contained within the interior of the long pipe. In this regard, in an embodiment, after the tissue for removal has been removed, the present methods further comprise depressing the suction button to activate the suction pipe, thereby removing any excess blood in the body of the patient at the surgical site. Similarly, in another embodiment, after the tissue for removal has been removed, the present methods further comprise depressing the irrigation button to activate the irrigation pipe, thereby irrigating the surgical site to clean out the surgical site and ensuring that infection risks are minimized in the body of the patient.

It is to be understood that multifunctional laparoscopic suction and irrigation surgical grasping method and device is not limited to the specific embodiments described above but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A multifunctional laparoscopic suction and irrigation surgical grasping tool, comprising:
a fixed handle including a suction button, an irrigation button, and a diathermic connector on a topside of the fixed handle;
a hollow long pipe including an interior, a distal end and a proximal end, the interior including a suction pipe with an intake hole and an irrigation pipe with a discharge hole;
a rotating grasper collar disposed on an end of the fixed handle connecting the fixed handle to the proximal end of the long pipe, wherein the rotating grasper collar is connected to the fixed handle with a bearing;
a grasper handle which is configured such that, when squeezed so that the grasper handle is moved closer to the fixed handle, the grasper handle produces a firing action; and
a grasper comprising grasping forceps having jaws, the grasper extending from the distal end of the long pipe and housed in a channel above the suction pipe and the irrigation pipe, wherein a rotation of the rotating grasper collar with respect to an axis of the long pipe causes the grasper to change an angular orientation of the grasper, wherein the grasping forceps are 5 mm apart when the jaws of the forceps are in an open V-shape configuration, and wherein when the firing action is produced by squeezing the grasper handle, the jaws of the forceps close shut around tissue and retract back towards the distal end of the long pipe.

2. (canceled)

3. The multifunctional laparoscopic suction and irrigation surgical grasping tool as recited in claim 1, wherein said grasper carries an electric charge.

4. (canceled)

5. The multifunctional laparoscopic suction and irrigation surgical grasping tool as recited in claim 1, wherein the fixed handle has an inverted L-shaped and a hollow structure.

6. The multifunctional laparoscopic suction and irrigation surgical grasping tool as recited in claim 5, wherein the grasper handle is connected to the fixed handle at a connection hub contained within the hollow structure of the fixed handle.

7. (canceled)

8. (canceled)

9. The multifunctional laparoscopic suction and irrigation surgical grasping tool as recited in claim 1, wherein the diathermic connector provides the grasper with an electric charge at a point of contact situated within the hollow structure of the fixed handle.

10. The multifunctional laparoscopic suction and irrigation surgical grasping tool as recited in claim 9, wherein a channel of the grasper is overlayed over the suction pipe and the irrigation pipe such that the channel of the grasper, the suction pipe, and the irrigation pipe are all contained within the interior of the long pipe.

11. A method of assisting in laparoscopic surgery in a patient, the method comprising:
making an incision in a surgical site in a body of the patient;
inserting a device having a grasper through the incision into the body of the patient, the device comprising:
a fixed handle including a suction button, an irrigation button, and a diathermic connector on a topside of the fixed handle;
a hollow long pipe including an interior, a distal end and a proximal end;
a rotating grasper collar disposed on an end of the fixed handle connecting the fixed handle to the proximal end of the long pipe, wherein the rotating grasper collar is connected to the fixed handle with a bearing;
a grasper handle; and
a grasper extending from the distal end of the long pipe, wherein a rotation of the rotating grasper collar with respect to an axis of the long pipe causes the grasper to change an angular orientation of the grasper;
squeezing the grasper handle to move the grasper handle closer to the fixed handle, thereby producing a firing action, wherein the firing action both activates an electric charge through the diathermic connector that is carried along the grasper and closes jaws of the forceps shut around tissue for removal in the body of the patient at the surgical site; and
retracting the grasper back towards the distal end of the long pipe, thereby removing the tissue for removal from the body of the patient at the surgical site;
wherein the electric charge carried along the grasper induces electric coagulation within tissue to remain in the body of the patient adjacent to the tissue for removal at the surgical site, thereby reducing tissue bleeding in the tissue to remain in the patient at the surgical site.

12. The method as recited in claim 11, wherein the device further comprises a suction pipe and an irrigation pipe within the long pipe, such that the grasper, the suction pipe, and the irrigation pipe are all contained within the interior of the long pipe.

13. The method as recited in claim 12, further comprising, after the tissue for removal has been removed, depressing the suction button to activate the suction pipe, thereby removing any excess blood in the body of the patient at the surgical site.

14. The method as recited in claim 12, further comprising, after the tissue for removal has been removed, depressing the irrigation button to activate the irrigation pipe, thereby irrigating the surgical site to clean out the surgical site and ensuring that infection risks are minimized in the body of the patient.
